(19) United States
(12) Patent Application Publication
(10) Pub. No.: US 2018/0350459 A1
(43) Pub. Date: Dec. 6, 2018

(54) METHODS AND APPARATUSES FOR IMPLEMENTING A SEMANTICALLY AND VISUALLY INTERPRETABLE MEDICAL DIAGNOSIS NETWORK

(71) Applicant: UNIVERSITY OF FLORIDA RESEARCH FOUNDATION, INC., Gainesville, FL (US)

(72) Inventor: Lin Yang, Lexington, KY (US)

(21) Appl. No.: 15/996,872

(22) Filed: Jun. 4, 2018

Related U.S. Application Data
(60) Provisional application No. 62/515,205, filed on Jun. 5, 2017.

(51) Int. Cl. G16H 15/00; G10L 15/183; G10L 15/16; G06K 9/62; G06N 3/08

(52) U.S. Cl. CPC G16H 15/00; G10L 15/183; G10L 15/16; G06K 2209/05; G06K 9/6232; G06N 3/08; G06K 9/6262

(57) ABSTRACT

Methods, systems, and apparatuses are provided for implementing a semantically and visually interpretable medical diagnosis network. An example method enables performance of medical diagnosis by a diagnostic system and comprises steps of receiving input comprising at least one of (i) a medical image and (ii) audio or text content, and inputting the received input into a deep learning framework of the diagnostic system. The example method further comprises steps of generating, by the deep learning framework, attention data regarding the received input, the attention data comprising pixel-level localized attention regions within the medical image, and generating, by the deep learning framework, a diagnostic report comprising (a) a diagnosis drawn based on the received input, the attention data, and corresponding audio or text content and (b) a medical justification of the diagnosis provided in a natural language format. Corresponding systems and apparatuses are contemplated herein.

### CROSS-REFERENCE TO RELATED APPLICATIONS

This application claims the benefit of U.S. Provisional Patent Application No. 62/515,205, filed Jun. 5, 2017, the entire contents of which are incorporated herein by reference.

### STATEMENT REGARDING FEDERALLY SPONSORED RESEARCH OR DEVELOPMENT

This invention was made with government support under R42 AG055375 awarded by the National Institute of Health. The government has certain rights in the invention.

### BACKGROUND

In recent years, the rapid development of deep learning technologies has shown remarkable impact on the biomedical image domain. Conventional image analysis tasks, such as segmentation and detection, support quick knowledge discovery from medical metadata to help manual diagnosis and decision-making. Automatic decision-making tasks, such as cancer stage and survival prediction, are usually treated as standard classification problems.

However, generic classification models are not an optimal solution for intelligent computer-aided diagnosis. Such models provide only brief conclusory diagnoses, and do not provide the rationales for their conclusions. In relying upon a generated diagnosis or decision-support system, a practitioner may need to understand the reasoning behind the generated diagnosis and may even require an interpretable explanation to provide to a patient or other practitioner of why a particular diagnosis or conclusion was reached. Current diagnostic systems are not equipped to produce interpretable medical justifications that support their decision-making process, so users of such systems may find it difficult to assess how accurately a diagnostic system captures and processes critical biomarker information. Without an understanding of how a diagnosis is determined, practitioners may have difficulty trusting or relying upon such computer-aided diagnosis systems.

### BRIEF SUMMARY

In general, embodiments of the present invention provide methods, apparatus, systems, computing devices, computing entities, and/or the like for implementing a semantically and visually interpretable medical diagnosis network. Moreover, example embodiments described herein provide a unified diagnostic system that can evaluate medical images, generate diagnostic reports, retrieve images by symptom descriptions, and visualize network attention, to provide justifications of the network diagnosis process.

In a first example embodiment, a method is provided for performing medical diagnosis by a diagnostic system. The method includes receiving input comprising at least one of (i) a medical image and (ii) audio or text content, inputting the received input into a deep learning framework of the diagnostic system, generating, by the deep learning framework, attention data regarding the received input, the attention data comprising pixel-level localized attention regions within the medical image, and generating, by the deep learning framework, a diagnostic report comprising (a) a diagnosis drawn based on the received input, the attention data, and corresponding audio or text content and (b) a medical justification of the diagnosis provided in a natural language format.

In some embodiments, the deep learning framework comprises an image model, a language model, and an auxiliary attention sharpening (AAS) module. The image model may comprise a residual network having residual blocks. In some such embodiments, the residual blocks are linked via ensemble-connection to overcome any gradient vanishing effect. The language model may comprise a long short-term memory (LSTM) network.

In some embodiments, inputting the received input into the deep learning framework of the diagnostic system comprises inputting the received input into the language model, and the method further comprises generating, by the image model and in response to the inputting the received input into the deep learning framework, a set of task tuples and a convoluted feature map. In some such embodiments, generating the attention data regarding the received input comprises inputting the convoluted feature map into the AAS module, and producing the attention data by the AAS module. To this end, generating the diagnostic report may comprise inputting the set of task tuples into the language model, producing, by the language model, a set of output language sequences, and generating the diagnostic report using the set of output language sequences and the attention data produced by the AAS module.

The method may further include training the deep learning framework. Training the deep learning framework may, in this regard, include inputting, to the diagnostic system, a plurality of medical images, each of the plurality of medical images comprising attention data comprising pixel-level localized attention regions within the medical image, inputting, to the diagnostic system, natural language components associated with corresponding portions of the attention data, and inputting, to the diagnostic system, one or more diagnostic reports, each diagnostic report comprising (i) a diagnosis drawn based on the medical image and the natural language components and (ii) a medical justification of the diagnosis provided in a natural language format, followed by training, by the diagnostic system, the deep learning framework using the medical image, the natural language components, and the one or more diagnostic reports.

In a second example embodiment, a diagnostic system is provided for performing medical diagnosis. The diagnostic system includes a communications interface configured to receive input comprising at least one of (i) a medical image and (ii) audio or text content, and a processing element configured to input the received input into a deep learning framework of the diagnostic system. The deep learning framework is configured to generate attention data regarding the received input, the attention data comprising pixel-level localized attention regions within the medical image, and to generate a diagnostic report comprising (a) a diagnosis drawn based on the received input, the attention data, and corresponding audio or text content and (b) a medical justification of the diagnosis provided in a natural language format.

In some embodiments, the deep learning framework comprises an image model, a language model, and an auxiliary attention sharpening (AAS) module. The image model may comprise a residual network having residual blocks, and in some cases the residual blocks are linked via ensemble-connection to overcome any gradient vanishing effect. The language model may comprise a long short-term memory (LSTM) network.

In some embodiments, inputting the received input into the deep learning framework of the diagnostic system comprises inputting the received input into the language model, and the image model is configured to generate, in response to the inputting the received input into the deep learning framework, a set of task tuples and a convoluted feature map. In some such embodiments, generating the attention data regarding the received input comprises inputting the convoluted feature map into the AAS module, and producing the attention data by the AAS module. In this regard, the deep learning framework may be configured to generate the diagnostic report by inputting the set of task tuples into the language model, producing, by the language model, a set of output language sequences, and generating the diagnostic report using the set of output language sequences and the attention data produced by the AAS module.

In some embodiments of the diagnostic system, the communications interface is further configured to receive a plurality of medical images, each of the plurality of medical images comprising attention data comprising pixel-level localized attention regions within the medical image, receive natural language components associated with corresponding portions of the attention data, and receive one or more diagnostic reports, each diagnostic report comprising (i) a diagnosis drawn based on the medical image and the natural language components and (ii) a medical justification of the diagnosis provided in a natural language format; and the processing element is further configured to train the deep learning framework using the medical image, the natural language components, and the one or more diagnostic reports.

In a third example embodiment, a computer program product is provided for performing medical diagnosis. The computer program product includes one or more non-transitory computer-readable storage medium storing instructions that, when executed, cause a diagnostic system to receive input comprising at least one of (i) a medical image and (ii) audio or text content, input the received input into a deep learning framework of the diagnostic system, generate, by the deep learning framework, attention data regarding the received input, the attention data comprising pixel-level localized attention regions within the medical image, and generate, by the deep learning framework, a diagnostic report comprising (a) a diagnosis drawn based on the received input, the attention data, and corresponding audio or text content and (b) a medical justification of the diagnosis provided in a natural language format.

In some embodiments, the deep learning framework comprises an image model, a language model, and an auxiliary attention sharpening (AAS) module. The image model may comprise a residual network having residual blocks, and in some cases the residual blocks are linked via ensemble-connection to overcome any gradient vanishing effect. The language model may comprise a long short-term memory (LSTM) network.

In some embodiments, inputting the received input into the deep learning framework of the diagnostic system comprises inputting the received input into the language model, and the instructions, when executed, cause the diagnostic system to generate, by the image model and in response to the inputting the received input into the deep learning framework, a set of task tuples and a convoluted feature map. In some such embodiments, generating the attention data regarding the received input comprises inputting the convoluted feature map into the AAS module, and producing the attention data by the AAS module. To this end, generating the diagnostic report may comprise inputting the set of task tuples into the language model, producing, by the language model, a set of output language sequences, and generating the diagnostic report using the set of output language sequences and the attention data produced by the AAS module.

The instructions, when executed, may further cause the diagnostic system to train the deep learning framework. Training the deep learning framework may, in this regard, include inputting, to the diagnostic system, a plurality of medical images, each of the plurality of medical images comprising attention data comprising pixel-level localized attention regions within the medical image, inputting, to the diagnostic system, natural language components associated with corresponding portions of the attention data, and inputting, to the diagnostic system, one or more diagnostic reports, each diagnostic report comprising (i) a diagnosis drawn based on the medical image and the natural language components and (ii) a medical justification of the diagnosis provided in a natural language format, followed by training, by the diagnostic system, the deep learning framework using the medical image, the natural language components, and the one or more diagnostic reports.

The above summary is provided merely for purposes of summarizing some example embodiments to provide a basic understanding of some aspects of the invention. Accordingly, it will be appreciated that the above-described embodiments are merely examples and should not be construed to narrow the scope or spirit of the invention in any way. It will be appreciated that the scope of the invention encompasses many potential embodiments in addition to those here summarized, some of which will be further described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Having thus described the invention in general terms, reference will now be made to the accompanying drawings, which are not necessarily drawn to scale.

FIG. 1 illustrates an overview of an example medical diagnosis network (MDNet) for generating interpretable diagnosis process.

FIGS. 2A and 2B provide high-level illustrations of one example embodiment described herein.

FIG. 3 illustrates a comparison of the qualitative results between two attention mapping methods described herein.

FIG. 4 is an overview of a system that can be used to practice embodiments of the present disclosure.

FIG. 5 is an exemplary schematic diagram of a computing entity according to one embodiment of the present disclosure.

FIGS. 6 and 7 provide flowcharts illustrating operations and processes that can be used in accordance with various embodiments of the present disclosure.

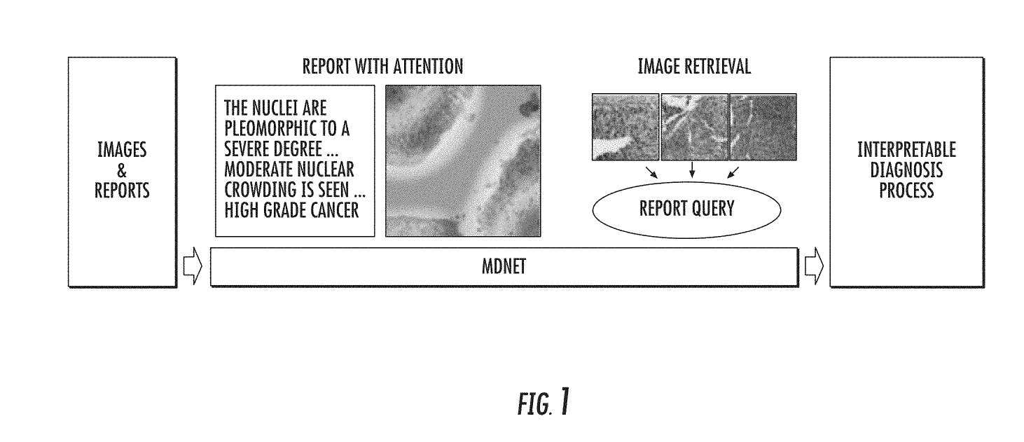

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout.

### I. COMPUTER PROGRAM PRODUCTS, METHODS, AND COMPUTING ENTITIES

Embodiments of the present invention may be implemented in various ways, including as computer program products that comprise articles of manufacture. A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### II. EXEMPLARY SYSTEM ARCHITECTURE

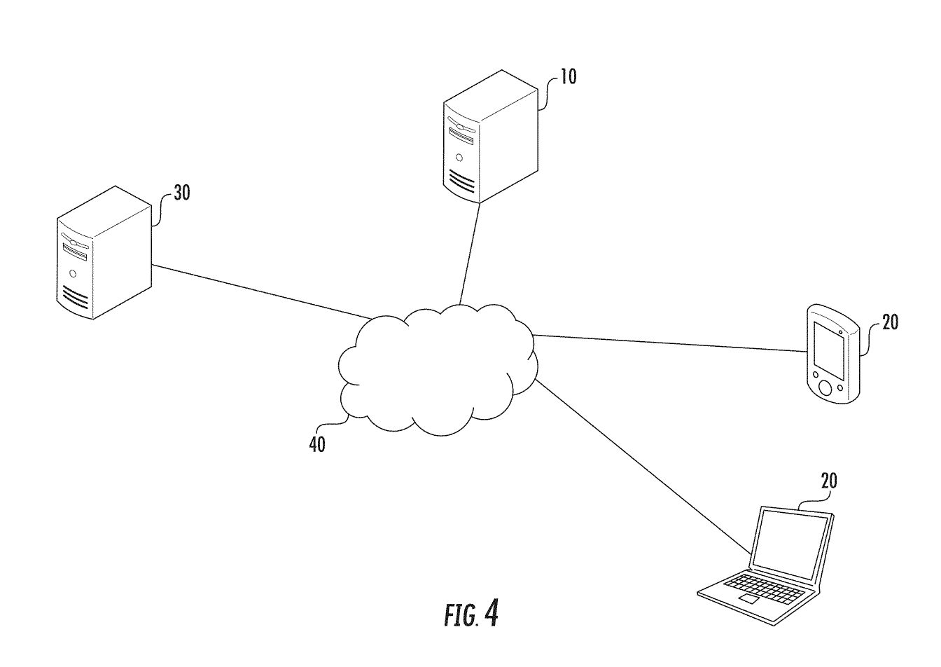

FIG. 4 provides an illustration of an exemplary embodiment of the present invention. As shown in FIG. 4, this particular embodiment may include one or more diagnostic computing entities 10, one or more user computing entities 20, one or more information/data computing entities 30, one or more networks 40, and/or the like. Each of these components, entities, devices, systems, and similar words used herein interchangeably may be in direct or indirect communication with, for example, one another over the same or different wired or wireless networks. Additionally, while FIG. 4 illustrates the various system entities as separate, standalone entities, the various embodiments are not limited to this particular architecture.

### 1. Exemplary Diagnostic Computing Entity

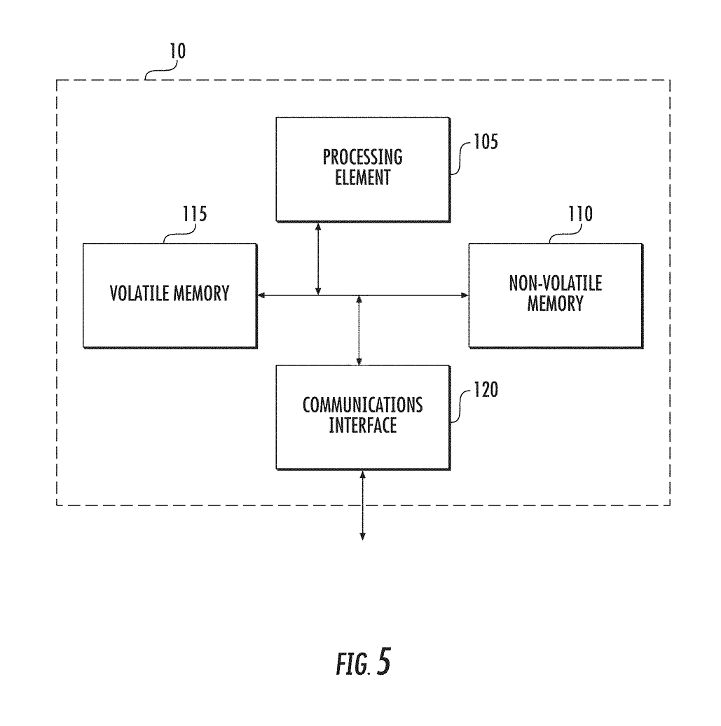

FIG. 5 provides a schematic of a diagnostic computing entity 10 according to one embodiment of the present disclosure. In example embodiments, a diagnostic computing entity 10 may be configured to train a deep learning framework (e.g., a deep neural network, such as a convolutional neural network or the like) for medical image predictive diagnosis. In other example embodiments, a diagnostic computing entity 10 may be configured to perform predictive diagnosis with a trained diagnostic system. In some examples, the diagnostic computing entity 10 may comprise or otherwise be in communication with a diagnostic system. The diagnostic computing entity 10 may be a server configured to communicate with the diagnostic system. The diagnostic system may also comprise any of the components described herein with respect to FIG. 5.

In general, the terms computing entity, computer, entity, device, system, and/or similar words used herein interchangeably may refer to, for example, one or more computers, computing entities, desktops, mobile phones, tablets, phablets, notebooks, laptops, distributed systems, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

In one embodiment, the diagnostic computing entity 10 may also include one or more communications interfaces 120 for communicating with various other computing entities, such as by communicating data, content, information, and/or similar terms used herein interchangeably that can be transmitted, received, operated on, processed, displayed, stored, and/or the like.

As shown in FIG. 5, in one embodiment, the diagnostic computing entity 10 may include or be in communication with one or more processing elements 105 (also referred to as processors, processing circuitry, and/or similar terms used herein interchangeably) that communicate with other elements within the diagnostic computing entity 10 via a bus, for example. As will be understood, the processing element 105 may be embodied in a number of different ways. For example, the processing element 105 may be embodied as one or more complex programmable logic devices (CPLDs), microprocessors, multi-core processors, co-processing entities, application-specific instruction-set processors (ASIPs), microcontrollers, and/or controllers. Further, the processing element 105 may be embodied as one or more other processing devices or circuitry. The term circuitry may refer to an entirely hardware embodiment or a combination of hardware and computer program products. Thus, the processing element 105 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 105 may be configured for a particular use or configured to execute instructions stored in volatile or non-volatile media or otherwise accessible to the processing element 105. As such, whether configured by hardware or computer program products, or by a combination thereof, the processing element 105 may be capable of performing steps or operations according to embodiments of the present disclosure when configured accordingly.

In one embodiment, the diagnostic computing entity 10 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 110, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the diagnostic computing entity 10 may further include or be in communication with volatile media (also referred to as volatile storage, memory, memory storage, memory circuitry and/or similar terms used herein interchangeably). In one embodiment, the volatile storage or memory may also include one or more volatile storage or memory media 115, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 105. Thus, the databases, database instances, database management systems, data, applications, programs, program modules, scripts, source code, object code, byte code, compiled code, interpreted code, machine code, executable instructions, and/or the like may be used to control certain aspects of the operation of the diagnostic computing entity 10 with the assistance of the processing element 105 and operating system.

As indicated, in one embodiment, the diagnostic computing entity 10 may also include one or more communications interfaces 120 for communicating with various other computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the diagnostic computing entity 10 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra wideband (UWB), infrared (IR) protocols, near field communication (NFC) protocols, Wibree, Bluetooth protocols, wireless universal serial bus (USB) protocols, and/or any other wireless protocol.

Although not shown in FIG. 5, the diagnostic computing entity 10 may also comprise a user interface (that can include a display coupled to a processing element). For example, the user interface may include or be in communication with one or more input elements, such as a keyboard input, a mouse input, a touch screen/display input, motion input, movement input, audio input, pointing device input, joystick input, keypad input, and/or the like. The diagnostic computing entity 10 may also include or be in communication with one or more output elements (not shown), such as audio output, video output, screen/display output, motion output, movement output, and/or the like. These input and output elements may include software components such as a user application, browser, graphical user interface, and/or the like to facilitate interactions with and/or cause display of information/data from the diagnostic computing entity 10, as described herein. The user input interface can comprise any of a number of devices or interfaces allowing the user computing entity 20 to receive data, such as a keypad (hard or soft), a touch display, voice/speech or motion interfaces, or other input device. In embodiments including a keypad, the keypad can include (or cause display of) the conventional numeric (0-9) and related keys (#, *), and other keys used for operating the user computing entity 20 and may include a full set of alphabetic keys or set of keys that may be activated to provide a full set of alphanumeric keys.

As will be appreciated, one or more of the components of the diagnostic computing entity may be located remotely from other components of the diagnostic computing entity 10, such as in a distributed system. Furthermore, one or more of these components may be combined with additional components to perform various functions described herein, and these additional components may also be included in the diagnostic computing entity 10. Thus, the diagnostic computing entity 10 can be adapted to accommodate a variety of needs and circumstances. As will be recognized, these architectures and descriptions are provided for exemplary purposes only and are not limiting to the various embodiments.

### 2. Exemplary Deep Learning Framework

In various embodiments, the diagnostic computing entity 10 may be configured to execute or otherwise access and utilize a deep learning framework facilitating performance of various example embodiments described herein. The deep learning network, which may be referred to herein as MDNet, can read images, generate diagnostic reports, retrieve images by symptom descriptions, and visualize network attention, to provide justifications of the network diagnosis process. Training MDNet requires use of a direct multimodal mapping from report sentence words to image pixels, which has historically been a significant problem because discriminative image features to support diagnostic conclusion inference is “latent” in reports rather than offered by specific image/object labels. Effectively utilizing these semantic information in reports, as described herein, thus produces a more effective methodology for image-language modeling.

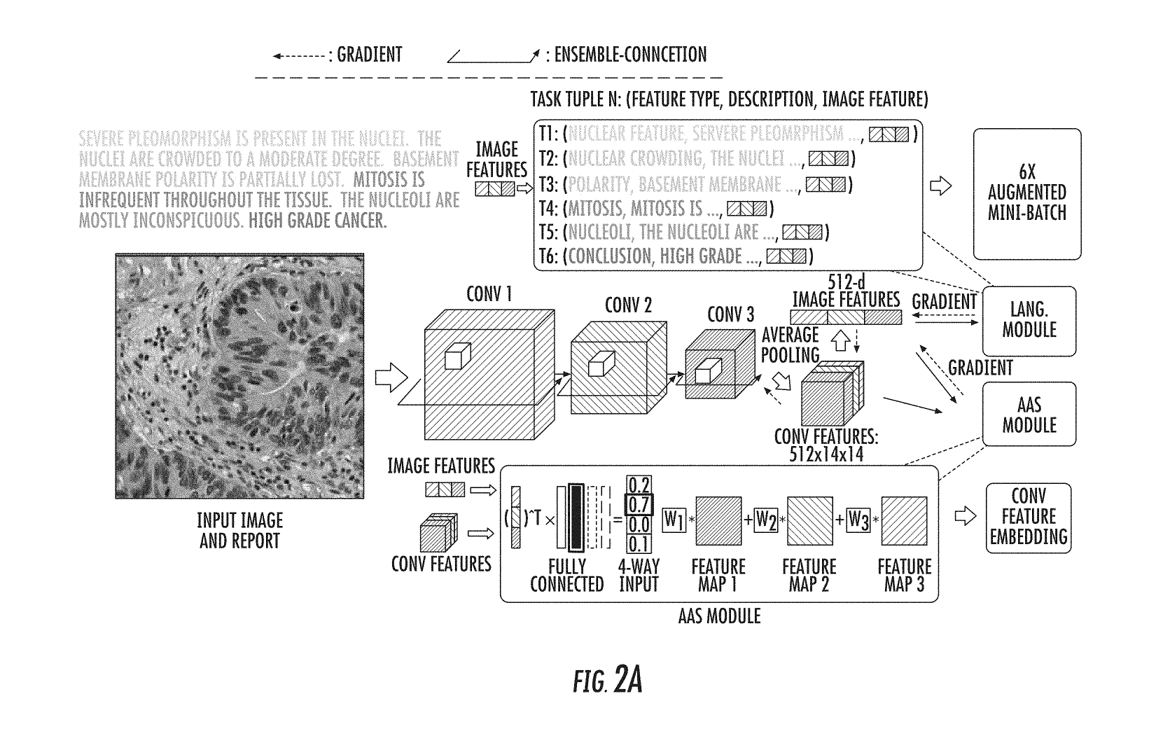

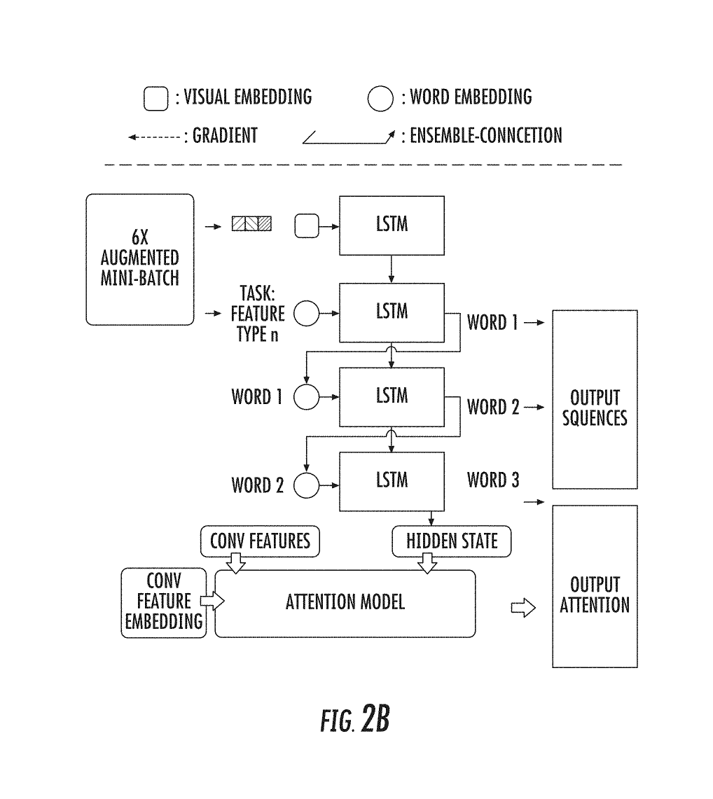

The deep learning framework may comprise an image model, a language model, and an auxiliary attention sharpening (AAS) module, and will be described below in connection with FIGS. 2, 3, 4, and 5. Each component of which is described herein. For image modeling based on convolutional neural networks (CNN), example embodiments provide the capability of the network to capture size-variant symptomatic image features (such as mitosis depicted in pixels or cell polarity depicted in regions) for image representations. Moreover, the image model overcomes the weakness of the residual network (ResNet) from the ensemble learning aspect by utilizing ensemble-connection to encourage multi-scale representation integration, which results in more efficient feature utilization. Language models contemplated herein may comprise Long Short-Term Memory (LSTM) networks. Moreover, as described herein, an optimization approach is used in some embodiments to train the overall network end-to-end starting from scratch. Finally, an attention mechanism is used to enhance the visual feature alignment of the image model with sentence words to obtain sharper attention maps.

Example embodiments of the image model are based on the well-known residual network (ResNet), which introduces identity mapping skip-connection to address network training difficulty. Identity mapping gradually becomes an acknowledged strategy to overcome the barrier of training very deep networks. Besides, skip-connection encourages the integration of multi-scale representations for more efficient feature utilization. In this regard, the identity mapping in the newest ResNet is a simple yet effective skip-connection to allow the unimpeded information flow inside the network. Each skip-connected computation unit is called a residual block. In a ResNet with L residual blocks, the forward output y_{L} from the l-th residual block and the gradient of the loss [symbol] with respect to its input y_{L} is defined as

$$\begin{matrix}{y}_{L} = {y}_{l} + {\sum }_{m = 1}^{L - 1} {ℱ}_{m} ( {y}_{m} ) , & ( 1 ) \\ \frac{\partial ℒ}{\partial {y}_{l}} = \frac{\partial ℒ}{\partial {y}_{L}} ( {\sum }_{m = 1}^{L - 1} {ℱ}_{m} ( {y}_{m} ) ) , & ( 2 )\end{matrix}$$

where [symbol] ₘ is composed by consecutive batch normalization, rectified linear units (ReLU), and convolution. Thanks to the addition scheme, the gradient

$$( i . e . , \frac{\partial ℒ}{\partial {y}_{L}} )$$

in backward can flow directly to preceding layers without passing through any convolutional layer. Since the weights of convolutional layers can scale gradients, this property alleviates the gradient vanishing effect when the depth of the network increases.

One skip-connection in a residual block offers two information flow paths, so the total path increases exponentially as network goes deeper. Recent work shows that ResNet with n residual blocks can be interpreted as the ensemble of 2ⁿ relatively shallow networks. It can be viewed that the exponential ensembles boost the network performance. Consequently, this viewpoint reveals a weakness of ResNet by probing its classification module.

In ResNet and other related networks, the classification module connecting convolutional layers includes a global average pooling layer and a fully connected layer. The two layers are mathematically defined as

$${P}^{c} = {\sum }_{k} {ω}_{k}^{c} \cdot {\sum }_{i , j} {y}_{L}^{( k )} ( i , j ) , \qquad ( 3 )$$

where P^{c} is the probability output of class c. (i,j) is spatial coordinates. ω^{c}=[ω₁^{c}, . . . , ωₖ^{c}, . . . ]^{T} is the c-th column of the weight matrix of the fully connected layer applied onto P^{C}. y_{L}^{(k)} is the k-th feature map of the last residual block. By plugging Eq. (1) into Eq. (3), one can see that P^{c} is the weighted average of the summed ensemble output:

$${P}^{c} = {\sum }_{i , j} {ω}^{c} {y}_{L} = {\sum }_{i , j} {ω}^{c} ( {y}_{1} + {\sum }_{m = 1}^{L - 1} {ℱ}_{m} ) . \qquad ( 4 )$$

However, using a single weighting function in the classification module is suboptimal in this situation. This is because the outputs of all ensembles share classifiers such that the importance of their individual features are undermined. To address this issue, embodiments of the present image model decouple the ensemble outputs and apply classifiers to them individually by using

$${P}^{c} = {\sum }_{i , j} ( {ω}_{1}^{c} \cdot {y}_{1} + {\sum }_{m = 1}^{L - 1} {ω}_{m + 1}^{c} \cdot {ℱ}_{m} ) . \qquad ( 5 )$$

The above equation assigns individual weight ω₁^{c} to ω_{L}^{c} for each ensemble output, which enables the classification module to independently decide the information importance from different ensembles.

Accordingly, an example image model contemplated herein includes a “redesign” of the ResNet architecture to realize the above idea, i.e., a new way to skip-connect a residual block, defined as follows:

yᵢ₊₁= [symbol] ₗ(yₗ)⊗ₗ (6)

where ⊗ is the concatenation operation. This skip-connection scheme is referred to herein as an ensemble connection. It allows outputs from residual blocks to flow through concatenated feature maps directly to the classification layer in parallel (see FIGS. 2A and 2B), such that the classification module assigns weights to all network ensemble outputs and maps them to the label space. This design also ensures unimpeded information flow to overcome the gradient vanishing effect. As shown in FIGS. 2A and 2B, as an image is input with its accompanying diagnostic report into the MDNet system, the diagnostic report is separated into tuples and then inserted into the LSTM model, while the image features are inserted into the attention model and then co-trained in order to generate not only the image attention model but also the associated diagnostic report.

By applying ensemble connection between residual blocks connecting block groups where the feature map dimension changes, example image models contemplated herein maintain the identity mapping for blocks inside a group. Ensemble connection in nature integrates multi-scale representations in the last convolution layer.

Application of ensemble connection between residual blocks connecting block groups where the feature map dimension changes may utilize the following procedure. As an initial matter, the image model may adopt a ResNet with bottleneck residual block as an architecture baseline. Adding the ensemble connection to the original architecture is straightforward process that can easily implemented. In one example, there are four convolutional (conv) groups having different feature map dimensions. The first two groups have 32×32 dimension, and the last two have 16×16 and 8×8 dimensions, respectively. Replacing the identity mapping with an ensemble connection between adjacent residual blocks connecting these four groups allows multi-scale representation integration such that all representations can contribute to the classification directly. A 1×1 shortcut deals with feature map size reduce and dimension increase. In this example implementation, the structure of a conv group is defined as follows:

$$[ \begin{matrix}1 \times 1 , & 16 \times c / 4 \times w \\ 3 \times 3 , & 16 \times c / 4 \times w \\ 1 \times 1 , & 16 \times c \times w\end{matrix} ] , [ \begin{matrix}1 \times 1 , & 16 \times c / 4 \times 2 w \\ 3 \times 3 , & 16 \times c / 4 \times 2 w \\ 1 \times 1 , & 16 \times c \times 2 w\end{matrix} ] \times N - 1$$

where the left part shows the first residual block and the right part show the rest N−1 residual blocks in a conv group (2 to 4). Group 1 has a single [3×3, 16] convolutional layer. N increases as the network depth increases. The block type is indicted in the bracket, i.e., [kernel height×kernel width, kernel depth]. c is a multiplier of the kernel depth, c={1, 2, 2} for conv group {2, 3, 4}, respectively. w determines the width of the network. Since ensemble connection doubles feature maps intrinsically, the width from the second residual block grows by a factor of 2, as indicated by 2w in brackets.

Turning next to the language model, a LSTM network may be used to model the diagnostic reports by maximizing the joint probability over sentences:

$$log p ( {x}_{0 : T} \vert I ; {θ}_{L} ) = {\sum }_{t = 0}^{T} log p ( {x}_{t} \vert I , {x}_{0 : t - 1} ; {θ}_{L} ) , \qquad ( 7 )$$

where {x₀, . . . , x_{T},} are sentence words (encoded as one-hot vectors). The LSTM parameters θ_{L} are used to compute several LSTM internal states. We integrate the “soft” attention mechanism into LSTM through a context vector zₜ (defined as follows), capturing localized visual information. To make a prediction, LSTM takes the output of the last time step xₜ₋₁ along with hidden state hₜ₋₁ and zₜ as inputs, and computes the probability of next word xₜ as follows:

hₜ₋₁=LTSM(Exₜ₋₁,hₜ₋₁,zₜ)

p(xₜ|l,x_{0:t-1};θ_{L}∝exp(Gₕhₜ)) (8)

where E is the word embedding matrix, and Gₕ decodes hₜ to the output space. The attention model dynamically computes a weight vector at to extract important image locations supporting the word prediction, which is interpreted as an attention map indicting where networks capture useful visual information. Attention is the main component supporting the visual interpretability of our network. In practice, however, the original attention mechanism is more difficult to train, which often generates attention maps that smoothly highlight the majority of image area.

To address this issue, example embodiments utilize an auxiliary attention sharpening (AAS) module to improve learning effectiveness. The attention mechanism can be viewed as a type of alignment between image space and language space. Improving such alignment can be achieved by adding supervision on attention maps by using image region-level labels (e.g., bounding boxes). In order to deal with datasets that do not have any region-level labels, a new method is needed. As contemplated herein, rather than putting direct supervision on at, example embodiments tackle this problem by utilizing the implicit class-specific localization property of global average pooling to generate informative Cony feature embeddings to support image-language alignment. Overall, zₜ can be computed as follows:

aₜ=softmax(Wₐₜₜ tan h(Wₕhₜ₁+c)),

c=(ω^{c})^{T}C(I),

zₜ=aₜC(I)^{T}, (9)

where Wₐₜₜ and Wₕ are learned embedding matrices, and C(I) is Conv feature maps (whose dimension is 512×(14·14)).

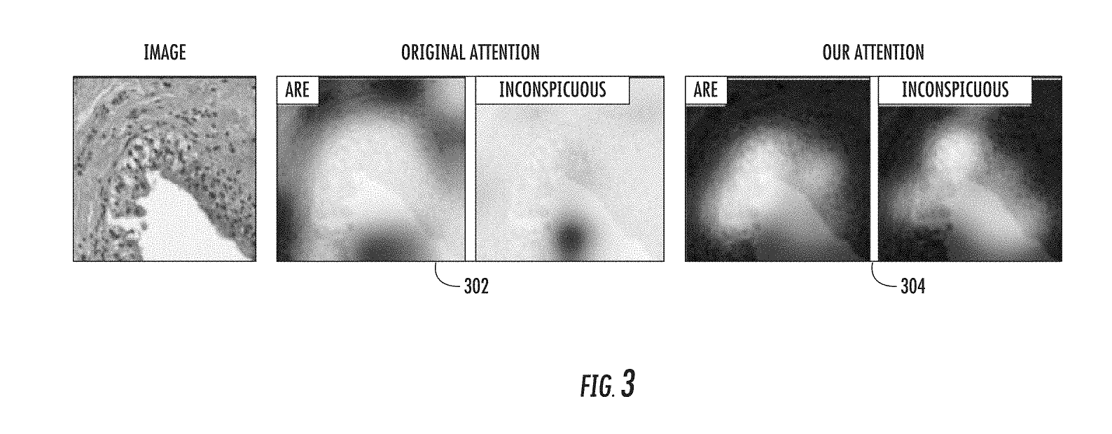

The original attention mechanism learns ω^{c} inside LSTM implicitly. In contrast, AAS adds an extra supervision to explicitly learn to provide more effective attention model training. Specifically, the formulation of this supervision is a revisit of Eq. (4), in which C(I) stands for y_{L}; (different notations are used for consistency). ω^{c} is a 512-dimensional vector corresponding to the c-th column of the fully connected weight matrix, selected by assigned class c (see FIGS. 2A and 2B); when applied to C(I), the obtained c that carries class-specific and localized region information is used to learn the alignment with hₜ₋₁ and compute a (14×14)-dimensional at and a 512-dimensional context vector zₜ. FIG. 3 compares the qualitative results between the original method and the method described herein.

In the well-known image captioning scheme, CNN provides an encoded image feature F(I) as the LSTM input x₀. Then a special START token is used as x₁ to inform the start of prediction. Generating effective gradients with respect to F(I) is the key for the image model optimization.

A complete medical diagnostic report describes multiple symptoms of observing images, followed by the diagnostic conclusion about either one or multiple type of diseases. For example, radiological images have multiple disease labels. Each symptom description specifically describes one type of image (symptom) feature. Effectively utilizing the semantic information in different descriptions is critical to generate effective gradient with respect to F(I) by LSTM.

As described herein, one LSTM may focus on mining discriminative information from a specific description. All description modeling shares LSTM. In this way, the modeling of each image feature description becomes a function of the complete report generation. We denote the number of functions as K. In the training stage, given a mini-batch with B pairs of image and reports, after forwarding the mini-batch to the image model, we duplicate each sample inside, resulting in a K×B mini-batch as the input of LSTM. Each duplication takes shared image information and one of K types particular feature description extracted from the report. The LSTM inputs of x₀^{e} and x₁^{e} for modeling image feature description task e is defined as

x₀^{e}=W_{F}F(I), x₁^{e}=ES(e), (10)

where W_{F} is a learned image feature embedding matrix. S(e), e={1, . . . , K} is the one-hot representation of the e-th image feature type. In this way, we use particular x₁^{e} to inform LSTM the start of a targeting task. During back-propagation, the gradient with respect to F(I) from duplications are merged. All the operations are end-to-end trainable.

In example embodiments, the diagnostic conclusions as labels for supervision of AAS. The motivation is two-fold. First, Cony feature embeddings generated by AAS are specific to conclusion labels; since all symptom descriptions support the inference of the diagnostic conclusion, Conv feature embeddings in nature contain useful visual information supporting different types of image feature descriptions and thereby facilitate better alignment with description words in the attention model. Second, AAS serves as a weak supervision for the image model, which makes sure the image model training towards to optimal diagnostic conclusion.

The overall model has three sets of parameters: θ_{D} in the image model D, θ_{L} in the language model L, and θ_{M} in the AAS module M. The overall optimization problem in MDNet is defined as

$${max}_{{θ}_{L} , {θ}_{D} , {θ}_{M}} {ℒ}_{M} ( {l}_{c} , M ( D ( I ; {θ}_{D} ) ; {θ}_{M} ) ) + {ℒ}_{L} ( {l}_{s} , L ( D ( I ; {θ}_{D} ) ; {θ}_{L} ) ) , \qquad ( 11 )$$

where {I, l_{c}, lₛ} is a training tuple: input image I, label l_{c} and ground truth report sentence lₛ (defined as follows). Modules M and L are supervised by two negative log-likelihood losses [symbol] _{M} and [symbol] _{L}, respectively.

The updating process of θ_{M} and θ_{L} is independent and straightforward using gradient descent. Updating θ_{D} involves the gradients from the two modules. Moreover, example embodiments utilize a backpropagation scheme to allow their composite gradients to co-adapt mutually. The gradients in this method are calculated based on a mixture of a recurrent generative network and a multilayer perceptron. Specifically, θ_{D}D is updated as follows:

$${θ}_{D} ← {θ}_{D} - λ \cdot ( ( 1 - β ) \cdot \frac{\partial {ℒ}_{M}}{\partial {θ}_{D}} + β \cdot η \frac{\partial {ℒ}_{L}}{\partial {θ}_{D}} ) , \qquad ( 12 )$$

where λ is the learning rate, and β dynamically regulates two gradients during the training process. Moreover, another factor η is introduced to control the scale of

$$\frac{\partial {ℒ}_{M}}{\partial {θ}_{D}} .$$

because

$$\frac{\partial {ℒ}_{L}}{\partial {θ}_{D}} ,$$

a often has a smaller magnitude than

$$\frac{\partial {ℒ}_{L}}{\partial {θ}_{D}}$$

### 3. Exemplary User Computing Entity

In various embodiments, a user computing entity 20 may be configured to exchange and/or store information/data with the diagnostic computing entity 10. For instance, the user computing entity 20 may be used by a user (e.g., a scientist, lab technician or the like) to provide information to the diagnostic computing entity 10 for directing the training, image processing, attention data generation, diagnostic report generation and/or the like performed by the diagnostic computing entity 10. For example, the user computing entity 20 may be used to provide training data, including but not limited to attention data relative to a medical image, medical justification of a diagnosis (e.g., in natural language format), and/or the like. The user computing entity 20 may additionally or alternatively receive information/data from the diagnostic computing entity 10 or an information/data computing entity 30. For example, a diagnostic report generated by the diagnostic computing entity 10 may be provided for display on the user computing entity 20.

In one embodiment, the user computing entity 20 may include one or more components that are functionally similar to those of the diagnostic computing entity 10 described above. For example, in one embodiment, each user computing entity 20 may include one or more processing elements (e.g., CPLDs, microprocessors, multi-core processors, co-processing entities, ASIPs, microcontrollers, and/or controllers), volatile and non-volatile storage or memory, one or more communications interfaces, and/or one or more user interfaces.

### 4. Exemplary Information/Data Computing Entity

In various embodiments, the information/data computing entity 30 may be configured to receive, store, and/or provide information/data comprising medical images, diagnostic reports, and/or other information/data that may be requested by any of a variety of computing entities.

In one embodiment, an information/data computing entity 30 may include one or more components that are functionally similar to those of the diagnostic computing entity 10, user computing entity 20, and/or the like. For example, in one embodiment, each information/data computing entity 30 may include one or more processing elements (e.g., CPLDs, microprocessors, multi-core processors, co-processing entities, ASIPs, microcontrollers, and/or controllers), volatile and non-volatile storage or memory, one or more communications interfaces, and/or one or more user interfaces.

### III. EXEMPLARY SYSTEM OPERATION

Example embodiments of the present disclosure address problems in computer-aided medical diagnosis. As mentioned above, example embodiments train a diagnostic system having deep learning framework to perform medical diagnosis. In some embodiments, the trained diagnostic system relies on a multimodal mapping of an image model to a language model relating to diagnostic reports.

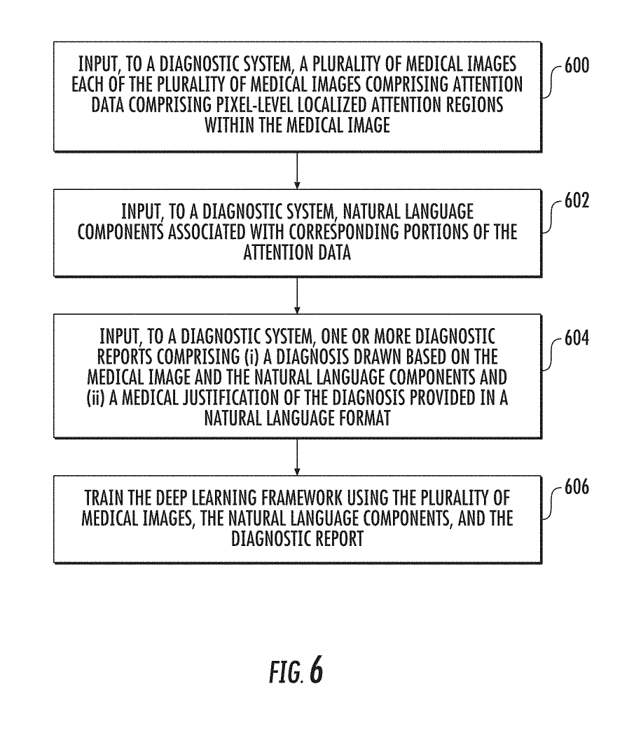

FIG. 6 illustrates a flowchart containing a series of operations for training the deep learning framework. The operations illustrated in FIG. 6 may, for example, be performed by, with the assistance of, and/or under the control of the diagnostic computing entity 10, as described above. In this regard, performance of the operations may invoke one or more of the processing element 105, memory (e.g., non-volatile memory 110 or volatile memory 115), or communications circuitry 120. The sequence of operations described in FIG. 6 provide one set of processes and procedures that may be performed in an example embodiment for training a deep learning framework for medical image predictive diagnosis, although it will be understood that other embodiments may perform more or fewer operations than the specific example outlined herein.

At block 600, the diagnostic computing entity 10 includes means, such as memory (e.g., non-volatile memory 110 or volatile memory 115), communications circuitry 120, or the like, for receiving a plurality of medical images. In this regard, each of the plurality of medical images may comprise attention data comprising pixel-level localized attention regions within the medical image. For example, a practitioner or other user may indicate on a sample medical image the regions of the image on which a diagnosis is based.

At block 602 the diagnostic computing entity 10 includes means, such as memory (e.g., non-volatile memory 110 or volatile memory 115), communications circuitry 120, or the like, for receiving inputs, to the diagnostic system, natural language components associated with corresponding portions of the attention data. The natural language components may be descriptions of the corresponding portions of the attention data. In this regard, these medical descriptions may be natural language and/or audio descriptions. For example, the natural language components may be provided verbally or in writing, and may be the descriptions a doctor or instructor provides to resident physicians and/or the like while describing how the diagnosis was determined. More specifically, these descriptions may explain the significance of the corresponding portions of the attention data. When provided as a set of text, the natural language components may include associations at the word-level or other semantic component to the attention data in the medical image. This may be achieved by a practitioner describing the rationale behind the diagnosis while pointing to specific regions of the medical image. During the training stage, each training image is connected with its associated diagnostic report and trained together within the deep learning framework, instead of training the framework separately. In this way, the diagnostic system can learn the association rules because the report and the digitized image during the entire training stage.

At block 604, the diagnostic computing entity 10 includes means, such as memory (e.g., non-volatile memory 110 or volatile memory 115), communications circuitry 120, or the like, for receiving one or more diagnostic reports, each diagnostic report comprising (i) a diagnosis drawn based on the medical image and the natural language components and (ii) a medical justification of the diagnosis provided in a natural language format. In some embodiments, the diagnosis and medical justification may be provided as training data and may represent expected outputs of the diagnosis system when processing the corresponding medical images and set of text.

At block 606, the diagnostic computing entity 10 includes means, such as processing element 105 or the like, for training the deep learning framework using the medical image, the natural language components, and the one or more diagnostic reports.

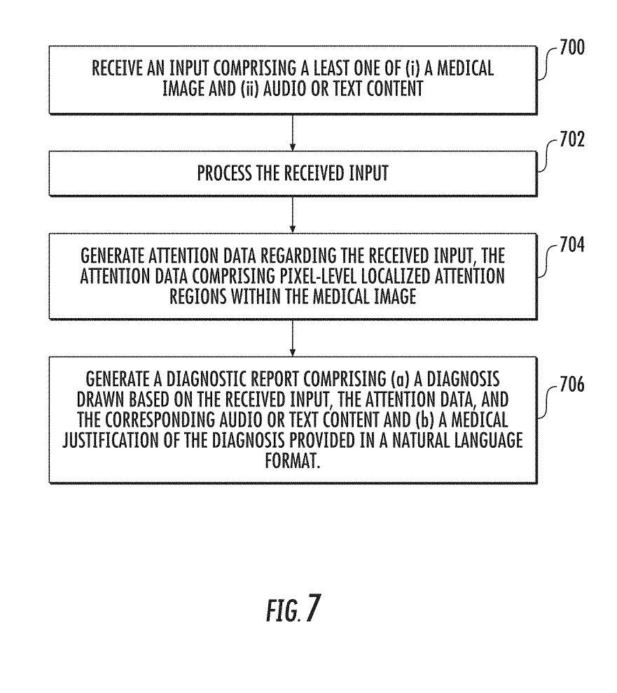

FIG. 7 provides another flowchart illustrating processes and procedures that may be performed by the diagnostic computing entity 10 to perform medical diagnosis by the trained diagnostic system. As with the operations described in connection FIG. 6, the operations illustrated in FIG. 7 may, for example, be performed by, with the assistance of, and/or under the control of the diagnostic computing entity 10, and performance of the operations may invoke one or more of the processing element 105, memory (e.g., non-volatile memory 110 or volatile memory 115), or communications circuitry 120. The sequence of operations described in FIG. 7 provide one set of processes and procedures that may be performed in an example embodiment for performing medical diagnosis, although it will be understood that other embodiments may perform more or fewer operations than the specific example outlined herein.

At block 700, the diagnostic computing entity 10 includes means, such as memory (e.g., non-volatile memory 110 or volatile memory 115), communications circuitry 120, or the like, for receives an input comprising at least one of a medical image and audio or language (e.g., text) content. When receiving audio input, the diagnostic computing entity 10 may further include means for converting the audio content into text content, such as speech recognition software executable by the processing element 105.

At block 702, the diagnostic computing entity 10 includes means, such as processing element 105 or the like, for processing the received input with the trained diagnostic system. In turn, processing the received input may comprise multiple sub-operations. As an initial matter, means for processing the received input may operate by inputting the received input into a deep learning framework of the diagnostic system. In this regard, it will be understood that the deep learning framework may comprise an image model, a language model, and an auxiliary attention sharpening (AAS) module. The image model, in turn may comprise a residual network having residual blocks. To this end, the residual blocks may be linked via ensemble-connection to overcome any gradient vanishing effect, as described above in greater detail. Moreover, the language model may comprise a long short-term memory (LSTM) network. In any event, inputting the received input into the deep learning framework of the diagnostic system may comprise inputting the received input into the language model. In response to the inputting the received input into the deep learning framework, the image model may generate a set of task tuples and a convoluted feature map.

At block 704, the diagnostic computing entity 10 includes means, such as processing element 105 or the like, for generating attention data regarding the received input. Generating this attention data may comprise inputting the convoluted feature map into the AAS module, and in response, producing the attention data by the AAS module. In this regard, the attention data may comprise pixel-level localized attention regions within the medical image.

Finally, at block 706, the diagnostic computing entity 10 includes means, such as processing element 105 or the like, for generating a diagnostic report comprising (a) a diagnosis drawn based on the received input, the attention data, and corresponding audio or text content and (b) a medical justification of the diagnosis provided in a natural language format. In this regard, generating the diagnostic report may comprise inputting the set of task tuples into the language model, which in response produces a set of output language sequences, and then generating the diagnostic report using the set of output language sequences and the attention data. The diagnostic report generated at block 706 may include a digitized image that contains all the region of interests (ROI). These computer generated ROIs contains important diagnostic clues that doctors will use for diagnosis. In addition, diagnostic report generated at block 706 may include will include not only a diagnosis, but a medical justification explaining, with reference to the attention data and/or medical image, the rationale which underpins the determination of the diagnosis.

The procedures described above have many practical applications. As described previously, current diagnostic systems are not equipped to produce interpretable medical justifications that support their decision-making process, so users of such systems may find it difficult to assess how accurately a diagnostic system captures and processes critical biomarker information. Without an understanding of how a diagnosis is determined, practitioners may have difficulty trusting or relying upon such computer-aided diagnosis systems. Accordingly, example embodiments described herein provide a meaningful advance in computer functionality insofar as they are able to generate diagnostic reports that provide both a diagnosis and a medical justification of the diagnosis.

FIGS. 6 and 7 thus illustrate flowcharts describing the operation of apparatuses, methods, and computer program products according to example embodiments contemplated herein. It will be understood that each flowchart block, and combinations of flowchart blocks, may be implemented by various means, such as hardware, firmware, processor, circuitry, and/or other devices associated with execution of software including one or more computer program instructions. For example, one or more of the operations described above may be implemented by an apparatus executing computer program instructions. In this regard, the computer program instructions may be stored by a memory of the diagnostic computing entity 10 (e.g., non-volatile memory 110 or volatile memory 115), and executed by a processing element 105 of the diagnostic computing entity 10. As will be appreciated, any such computer program instructions may be loaded onto a computer or other programmable apparatus (e.g., hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture, the execution of which implements the functions specified in the flowchart blocks. The computer program instructions may also be loaded onto a computer or other programmable apparatus to cause a series of operations to be performed on the computer or other programmable apparatus to produce a computer-implemented process such that the instructions executed on the computer or other programmable apparatus provide operations for implementing the functions specified in the flowchart blocks.

The flowchart blocks support combinations of means for performing the specified functions and combinations of operations for performing the specified functions. It will be understood that one or more blocks of the flowcharts, and combinations of blocks in the flowcharts, can be implemented by special purpose hardware-based computer systems which perform the specified functions, or combinations of special purpose hardware with computer instructions.

### IV. CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for performing medical diagnosis by a diagnostic system, the method comprising:
receiving input comprising at least one of (i) a medical image and (ii) audio or text content;
inputting the received input into a deep learning framework of the diagnostic system;
generating, by the deep learning framework, attention data regarding the received input, the attention data comprising pixel-level localized attention regions within the medical image; and
generating, by the deep learning framework, a diagnostic report comprising (a) a diagnosis drawn based on the received input, the attention data, and corresponding audio or text content and (b) a medical justification of the diagnosis provided in a natural language format.

2. The method of claim 1, wherein the deep learning framework comprises an image model, a language model, and an auxiliary attention sharpening (AAS) module.

3. The method of claim 2, wherein the image model comprises a residual network having residual blocks.

4. The method of claim 3, wherein the residual blocks are linked via ensemble-connection to overcome any gradient vanishing effect.

5. The method of claim 2, wherein the language model comprises a long short-term memory (LSTM) network.

6. The method of claim 2,
wherein inputting the received input into the deep learning framework of the diagnostic system comprises inputting the received input into the language model, and
wherein the method further comprises generating, by the image model and in response to the inputting the received input into the deep learning framework, a set of task tuples and a convoluted feature map.

7. The method of claim 6, wherein generating the attention data regarding the received input comprises:
inputting the convoluted feature map into the AAS module; and
producing the attention data by the AAS module.

8. The method of claim 7, wherein generating the diagnostic report comprises:
inputting the set of task tuples into the language model;
producing, by the language model, a set of output language sequences; and
generating the diagnostic report using the set of output language sequences and the attention data produced by the AAS module.

9. The method of claim 1, further comprising:
training the deep learning framework.

10. The method of claim 9, wherein training the deep learning framework comprises:
inputting, to the diagnostic system, a plurality of medical images, each of the plurality of medical images comprising attention data comprising pixel-level localized attention regions within the medical image;
inputting, to the diagnostic system, natural language components associated with corresponding portions of the attention data;
inputting, to the diagnostic system, one or more diagnostic reports, each diagnostic report comprising (i) a diagnosis drawn based on the medical image and the natural language components and (ii) a medical justification of the diagnosis provided in a natural language format; and
training, by the diagnostic system, the deep learning framework using the medical image, the natural language components, and the one or more diagnostic reports.

11. A diagnostic system for performing medical diagnosis, the diagnostic system comprising:
a communications interface configured to receive input comprising at least one of (i) a medical image and (ii) audio or text content; and
a processing element configured to input the received input into a deep learning framework of the diagnostic system,
wherein the deep learning framework is configured to
generate attention data regarding the received input, the attention data comprising pixel-level localized attention regions within the medical image, and
generate a diagnostic report comprising (a) a diagnosis drawn based on the received input, the attention data, and corresponding audio or text content and (b) a medical justification of the diagnosis provided in a natural language format.

12. The diagnostic system of claim 11, wherein the deep learning framework comprises an image model, a language model, and an auxiliary attention sharpening (AAS) module.

13. The diagnostic system of claim 12, wherein the image model comprises a residual network having residual blocks.

14. The diagnostic system of claim 13, wherein the residual blocks are linked via ensemble-connection to overcome any gradient vanishing effect.

15. The diagnostic system of claim 12, wherein the language model comprises a long short-term memory (LSTM) network.

16. The diagnostic system of claim 12,
wherein inputting the received input into the deep learning framework of the diagnostic system comprises inputting the received input into the language model, and
wherein the image model is configured to generate, in response to the inputting the received input into the deep learning framework, a set of task tuples and a convoluted feature map.

17. The diagnostic system of claim 16, wherein generating the attention data regarding the received input comprises:
inputting the convoluted feature map into the AAS module; and
producing the attention data by the AAS module.

18. The diagnostic system of claim 17, wherein the deep learning framework is configured to generate the diagnostic report by:
inputting the set of task tuples into the language model;
producing, by the language model, a set of output language sequences; and
generating the diagnostic report using the set of output language sequences and the attention data produced by the AAS module.

19. The diagnostic system of claim 11,
wherein the communications interface is further configured to
receive a plurality of medical images, each of the plurality of medical images comprising attention data comprising pixel-level localized attention regions within the medical image,
receive natural language components associated with corresponding portions of the attention data, and
receive one or more diagnostic reports, each diagnostic report comprising (i) a diagnosis drawn based on the medical image and the natural language components and (ii) a medical justification of the diagnosis provided in a natural language format; and
wherein the processing element is further configured to train the deep learning framework using the medical image, the natural language components, and the one or more diagnostic reports.

20. A computer program product for performing medical diagnosis, the computer program product comprising one or more non-transitory computer-readable storage medium storing instructions that, when executed, cause a diagnostic system to:
receive input comprising at least one of (i) a medical image and (ii) audio or text content;
input the received input into a deep learning framework of the diagnostic system;
generate, by the deep learning framework, attention data regarding the received input, the attention data comprising pixel-level localized attention regions within the medical image; and
generate, by the deep learning framework, a diagnostic report comprising (a) a diagnosis drawn based on the received input, the attention data, and corresponding audio or text content and (b) a medical justification of the diagnosis provided in a natural language format.
